(19) United States
(12) Patent Application Publication
(10) Pub. No.: US 2007/0026456 A1
(43) Pub. Date: Feb. 1, 2007

(54) Method of biomolecule analysis and method of identifying biomolecule therewith

(75) Inventors: Kenichi Kamijo, Tokyo (JP); Hisao Kawaura, Tokyo (JP); Toru Sano, Tokyo (JP); Yo Tabuse, Tokyo (JP); Hirotaka Minagawa, Tokyo (JP); Kenji Miyazaki, Tokyo (JP)

Correspondence Address: YOUNG & THOMPSON, 745 SOUTH 23RD STREET, 2ND FLOOR, ARLINGTON, VA, 22202, US

(73) Assignee: NEC CORPORATION, Tokyo (JP)

(21) Appl. No.: 10/570,205

(22) PCT Filed: Sep. 2, 2004
(86) PCT No.: PCT/JP04/12752

§ 371 Date: Mar. 2, 2006

(30) Foreign Application Priority Data
Sep. 2, 2003 (JP) 2003-309682

(51) Int. Cl. G01N 33/53

(52) U.S. Cl. 435/7.1

(57) ABSTRACT

A biomolecule is analyzed with high accuracy. A biomolecule is analyzed with high likelihood. A biomolecule as an analysis subject is modified with a marker binding or adsorbing to only its specific portion (S101). Next, the biomolecule modified with the marker is developed on a base plate (S102). The arrangement of the biomolecule on the base plate is detected using a marker (S103). Then, scanning is performed along the shape of the biomolecule present on the detected position (S104). The biomolecule is analyzed based on an information relating to the shape or arrangement of the biomolecule obtained by scanning or an information relating to the arrangement of the marker on the biomolecule (S105).

### TECHNICAL FIELD

The present invention relates to a method of analyzing a biomolecule and a method of identifying a biomolecule using the same.

### BACKGROUND ART

In proteome analysis which analyzes the whole protein produced in specific cells, tissue or organs, an unknown protein present in cells and the like should be identified.

Conventionally, identification of a protein included in a proteome is effected by separating a protein by a two dimensional electrophoresis method and subjecting the separated protein to mass spectrometry. Here, in mass spectrometry of a high molecule substance such as a protein and the like, it is necessary to lower the molecular weight of a subject to be measured by enzymatic digestion. For this purpose, the separated protein is digested with an enzyme, and a purified peptide fragment is subjected to mass spectrometry. There is an investigation on a method of improving accuracy of such identification of a protein by mass spectrometry (Patent Document 1).

In the method of identification using mass spectrometry, however, repeatability is not obtained in production of a peptide fragment by enzymatic treatment, or back ground increases by remaining of an enzyme, in some cases. Further, detection sensitivity is around 10 femto mol, consequently, it is difficult to detect and identify a trace amount of protein with high sensitivity. Therefore, a technology which detects and analyzes a trace amount of protein with high sensitivity has been desired. [Patent Document 1] Japanese Laid-open patent publication NO. 11-94837

### DISCLOSURE OF THE INVENTION

The present invention has been accomplished in view of the above-mentioned conditions and has an object of providing a technology which analyzes a biomolecule with high accuracy. Another object of the present invention is to provide a technology analyzing the biomolecule with high likelihood.

According to the present invention, there is provided a method of analyzing a biomolecule, including elongating on a base plate a biomolecule having a specific portion selectively modified by a marker, detecting the a marker to specify a position of the elongated biomolecule on the base plate, and analyzing an arrangement of the marker in the biomolecule.

The analysis method according to the present invention detects a position of a biomolecule on a base plate using a marker. Thus, the biomolecule may be detected assuredly in one molecule unit. Since the biomolecule is elongated on a base plate, the modification position of a marker on the biomolecule may be infallibly analyzed. Because of this, it becomes possible to analyze a specific modification portion on the biomolecule with high accuracy and high likelihood.

In the method of analyzing a biomolecule of the present invention, the analyzing the arrangement of a marker may include measuring an interval of a plurality of the markers on the biomolecule and analyzing the arrangement of the marker. By this procedure, measurement results relating to an interval of markers may be utilized suitably for analysis of the arrangement of a marker. As a result, the biomolecule may be analyzed with higher accuracy and higher likelihood.

In the method of analyzing a biomolecule of the present invention, the elongating the biomolecule on a base plate includes immobilizing the biomolecule on the base plate and washing a surface of the base plate on which the biomolecule has been immobilized. By this procedure, unnecessary substances on a base plate or the biomolecule may be removed by washing. Accordingly, the arrangement of a marker may be analyzed with further higher accuracy and higher sensitivity.

In the method of analyzing a biomolecule of the present invention, the elongating the biomolecule on the base plate may include applying low frequency electric field on the base plate. By this procedure, the biomolecule may be elongated assuredly on a surface of the base plate.

In the method of analyzing a biomolecule of the present invention, the marker may be a fluorescent substance. Thus, the position of the biomolecule on the base plate may be detected with further higher sensitivity. Therefore, the biomolecule may be detected assuredly in one molecule unit.

In the method of analyzing a biomolecule of the present invention, the specifying the position of the biomolecule on a base plate may include irradiating a surface of the base plate with a light. By this procedure, the biomolecule may be detected further infallibly.

In the method of analyzing a biomolecule of the present invention, the analyzing the arrangement of a marker may include measuring irregularity on a surface of the biomolecule elongated on the base plate, to specify the modified position of the marker. By measuring irregularity on the surface of the biomolecule, the modification position of a marker in the biomolecule elongated on a base plate may be analyzed with high accuracy and high likelihood.

In the method of analyzing a biomolecule of the present invention, the irregularity may be measured by an atomic force microscope or a scanning tunnel microscope. Thus, irregularity on a surface of the biomolecule may be measured with high sensitivity.

The method of analyzing a biomolecule of the present invention may further include separating the biomolecule, prior to the elongating the biomolecule on the base plate. By this procedure, a predetermined biomolecule contained in a sample may be analyzed infallibly.

In the method of analyzing a biomolecule of the present invention, the biomolecule is a protein or a polypeptide, and the marker may modify a specific amino acid residue of the biomolecule. By this procedure, the arrangement information of a specific amino acid residue in a protein or a polypeptide elongated on a base plate may be obtained based on the arrangement information of a marker. Because of this, a protein or a polypeptide may be analyzed by a simple operation.

In the analysis method of the present invention, the marker may be a fluorescent substance which selectively modifies a lysine residue or a cysteine residue of the biomolecule. Thus, the arrangement information of a lysine residue or a cysteine residue of the biomolecule elongated on a base plate may be obtained. Because of this, the biomolecule may be analyzed with further higher accuracy and higher sensitivity.

The method of analyzing a biomolecule of the present invention may include modifying the biomolecule, prior to the elongating the biomolecule on the base plate. By this procedure, the biomolecule may be elongated on a base plate further assuredly.

In the method of analyzing a biomolecule of the present invention, the marker includes two or more fluorescent substances of different size, and these substances may modify selectively different amino acid residues in the biomolecule, respectively. By this procedure, wider information may be obtained regarding the arrangement of a marker on the biomolecule. Because of this, the accuracy and likelihood of analysis may be further improved.

According to the present invention, a method of identifying a biomolecule is provided, including analyzing the biomolecule by the aforementioned method of analyzing the biomolecule, then, further identifying the biomolecule based on information relating to the arrangement of the marker.

In the method of identify a biomolecule according to the present invention, the arrangement of a marker may be analyzed with high accuracy and high sensitivity since analysis is conducted using the analysis method. In the present invention, identification with excellent accuracy and sensitivity is made possible since the biomolecule is identified using information obtained by this analysis.

Any combinations of the constituent elements and embodiments including constituent elements and expressions of the present invention substituted mutually between methods and units are also effective as embodiments of the present invention.

According to the present invention, a biomolecule may be analyzed with high accuracy. Further, according to the present invention, the biomolecule may be analyzed with high likelihood.

### BRIEF DESCRIPTION OF THE DRAWINGS

The object, other object, features and advantages will be further clarified by preferable embodiments described below and appended drawings described below.

FIG. 1 is a view showing a procedure of a method of analyzing a biomolecule according the present embodiment.

FIG. 2 is a view illustrating modification of a biomolecule according to the present embodiment.

FIG. 3 is a view for illustrating the procedure of FIG. 1 in detail.

FIG. 4 is a view for illustrating developing of a biomolecule according to the present embodiment.

FIG. 5 is a view for illustrating elongation of a biomolecule according to the present embodiment.

FIG. 6 is a view for illustrating elongation of a biomolecule according to the present embodiment.

FIG. 7 is a view for illustrating elongation of a biomolecule according to the present embodiment.

FIG. 8 is a view for illustrating a procedure of a method of analyzing a biomolecule according to the present embodiment.

FIG. 9 is a view for illustrating the procedure of FIG. 8 in detail.

FIG. 10 is a view for illustrating the procedure of FIG. 8 in detail.

FIG. 11 is a view for illustrating the procedure of FIG. 8 in detail.

### BEST MODE FOR CARRYING OUT THE INVENTION

Embodiments of the present invention will be illustrated with reference to drawings. In all the drawings, same constituent elements are endowed with same symbols, and explanations thereof will not be appropriately repeated here.

(First Embodiment)

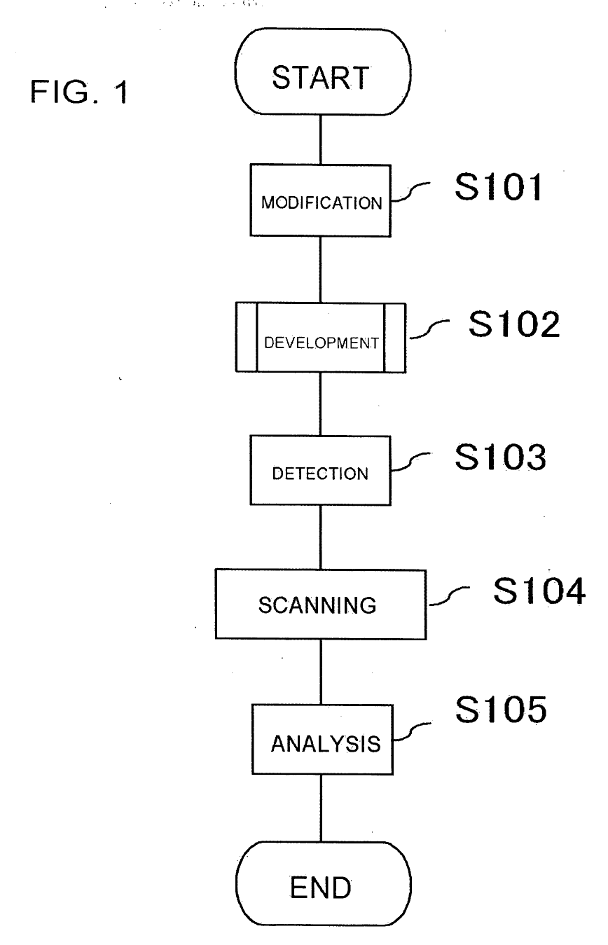

FIG. 1 is a view showing a procedure of a method of analyzing a biomolecule according to the present embodiment. In a flow in FIG. 1, a biomolecule as an analysis subject is first modified with a marker which binds or adsorbs only its specific portion (S101). The biomolecule modified with the marker is developed on a base plate (S102). On which position of the base plate the biomolecule is placed is detected with using a marker (S103). Then, scanning is performed along the shape of the biomolecule present in the detected position (S104). The biomolecule is analyzed based on information relating to the shape or arrangement of the biomolecule obtained by scanning or information relating to the arrangement of the marker on the biomolecule (S105)

In this embodiment, the biomolecule may be, for example, a protein or a polypeptide, a nucleic acid, a polysaccharide, a lipid or the like. Fragments thereof may also be permissible. Hereinafter, a case where analysis of a protein is performed will be exemplified.

In the flow of FIG. 1, the marker modifying a biomolecule in the step 101 is a substance which selectively modifies a specific region of the biomolecule. When the biomolecule is a protein, the marker specifically modifies, for example, a specific amino acid residue. The size of the marker is not particularly restricted as long as it is a size by which the position on the modified molecule may be specified in the step 104 described later.

The marker modifying one amino acid residue of a protein may be single, or several types of markers may be used. The marker may be a fine particle of metal, polymer and the like. When a single type of marker modifying one amino acid residue of a protein is used, the size or the shape of a plurality of markers modifying a biomolecule may be equalized. As a result, analysis accuracy of a biomolecule may be improved. Further, operation of scanning (step 104) may be made easier. When the marker is constituted of one type of molecule, its molecular weight may be, for example, about 300 to 1000.

As the marker, for example, fluorescent substances may be utilized. By using a fluorescent substance as the marker, the presence of a modified biomolecule may be detected easily at molecular level in the step 103 described later.

As the substance modifying a thio1 group in a biomolecule, there may be used, for example, compounds obtained by binding various fluorescent dyes to alkyl halides, maleimide, allylidyne or the like. By using these compounds, for example, a thioether stable under a condition of a pH of about 8 or less may be formed. Therefore, a fluorescent dye may be bounded selectively to a cysteine residue in a protein by using these compounds. As specific compounds, N-9-acridinylmaleimide, and Oregon Green 488 Iodoacetamide manufactured by Molecular Probes INC, and the like, for example, may be used.

As the substance modifying an amino group in a biomolecule, for example, compounds obtained by binding various fluorescent dyes to succinimide ester, sulfonyl chloride, dichlorotriazine and the like may be used. By using these compounds, a fluorescent dye may be selectively bound to a lysine residue in a protein. When the N-terminal of a protein as an analysis subject is free, the N-terminal of a protein may be modified simultaneously by modifying an amino group. Because of this, the N-terminal and the C-terminal of a protein may be discriminated in scanning (S104) and analysis (S105) described later. As a result, analysis accuracy and likelihood may be further improved of the above-mentioned modification substances for an amino group, a succinimide ester is preferably used since it may improve specificity for an amino group. As specific compounds, 2′,7′-Difluorofluorescein carboxylic acid succinimidyl ester, 5-Carboxyfluorescein diacetate-N-hydroxysuccinimide ester, and the like, for example, may be used.

As other fluorescent substances, for example, FITC (fluorescein isothiocyanate) derivatives, DANSYL (dimethylaminonaphthalenesulfonic acid) derivatives, fluorescamine, o-phthalaldehyde, and the like may also be used.

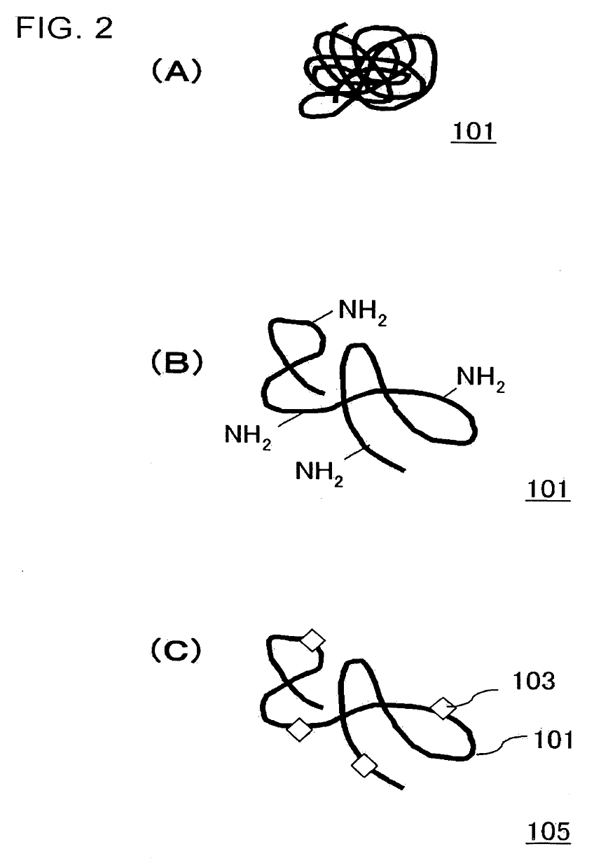

Fluorescent labeling of a protein may be conducted using a known method depending on the type of a marker. A protein may be, after previous denaturation, mixed with a marker. FIGS. 2A to 2C are views schematically showing a procedure which modifies an amino group of a protein. FIG. 2A is a view showing an native protein 101. When the protein 101 is unfolded in a buffer containing, for example, urea and surfactant (FIG. 2B) and mixed under this condition with a marker 103, the marker 103 may be modified assuredly to an exposed amino group (FIG. 2C).

By this procedure, a marker may be adsorbed or bound to a specific amino acid residue further assuredly, and resultantly, analysis accuracy may be improved. A remaining marker which is not used in modification of a protein may be removed by a method such as dialysis.

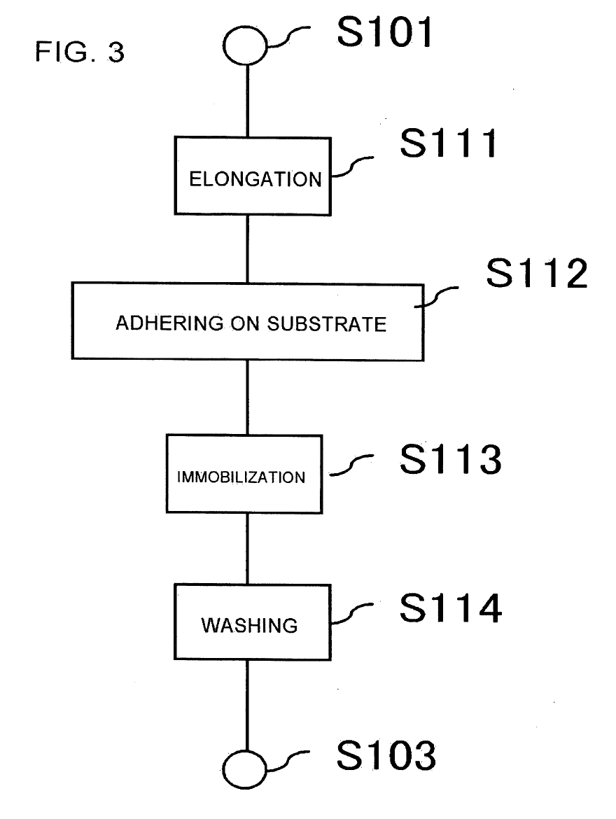

Spreading in the step 102 may be conducted, for example, according to the following procedure. FIG. 3 is a view illustrating in detail a procedure of the step 102 in FIG. 1. In FIG. 3, first, a protein modified with a marker is elongated or straightened (S111) and adhered on a base plate (S112). Under the elongated condition, the modified protein is immobilized on a base plate (S113). Thereafter, the surface of a base plate carrying the immobilized and modified protein is washed with water and the like (S114) to remove other substances remaining on the base plate.

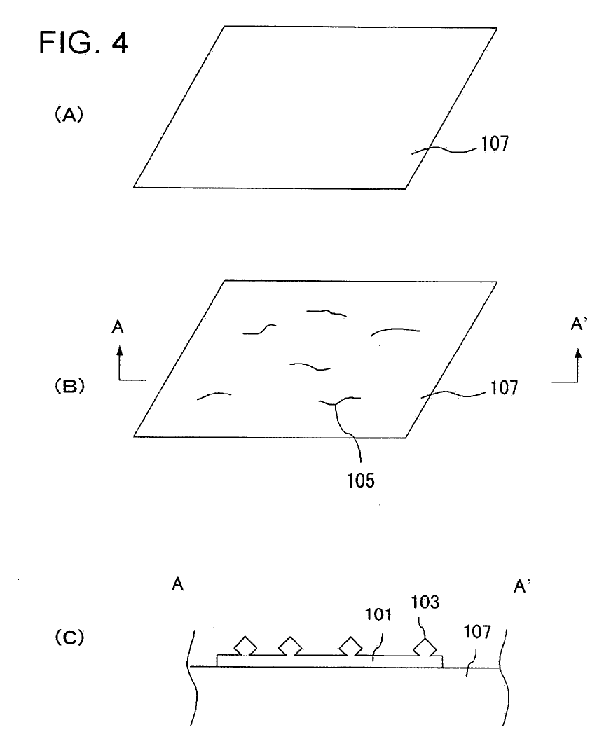

FIGS. 4A to 4B are views showing schematically a condition of developing of a protein on a base plate. FIG. 4A is a view showing a base plate 107. FIG. 4B is a view showing conditions (S111 to 112) of elongation of a modified protein 105 on the base plate 107. FIG. 4C is an enlarged cross-sectional view along A-A′ direction of FIG. 4B.

The material of the base plate 107 is constituted of silicon, glass, quartz, various plastic materials, or elastic materials such as a rubber. As the plastic material, materials which are easily molded are preferably used, and examples thereof include thermoplastic resins such as PMMA (polymethyl methacrylate), PET (polyethylene terephthalate), PC (polycarbonate), and thermosetting resins such as epoxy resins.

In analysis of the protein 101, it is preferable that the surface of the base plate 107 has hydrophobicity of an extent by which a protein adsorbs irreversibly. By this, immobilization of the modified protein 105 can be conducted easily. For example, predetermined hydrophobicization treatment may be performed on the surface of the base plate 107. As the base plate 107, glass may be used. When the base plate 107 is made of glass, a developed protein may be immobilized on the surface by an easy operation which will be described later. The surface of the base plate 107 may be hydrophobic. In this case, the modified protein 105 may be immobilized by introducing an immobilization reagent on the surface of the base plate 107, which will be described later.

The surface of the base plate 107 may also be coated with a metal such as gold (Au) It is preferable that the surface of the base plate 107 is kept under clean condition. When the base plate 107 is constituted of silicon, the surface of the base plate 107 may also be coated with a silicon oxide film (SiO₂)

Elongation of the modified protein 105 in the steps 111 to 112 may be conducted, for example, by adhering the modified protein 105 under condition of application of low frequency electric field to the base plate 107. Here, the low frequency electric field may be an electric field of 100 Hz or less. By this, the modified protein 105 in the form of random coil may be elongated along a certain direction (FIG. 4B).

For elongating the modified protein 105, the modified protein 105 may also be introduced on the base plate 107 under condition of application of high electric field to the base plate 107. Here, the high electric field may be an electric field of, for example, 500 kHz or more. In accordance therewith, the modified protein 105 may be elongated.

Here, adhesion of the modified protein 105 may be conducted, for example, by applying a liquid including the modified protein 105 on a base plate. Application may be, for example, spray application. Adhesion may also be conducted by a method in which a base plate is immersed in a liquid including the modified protein 105 and lifted. It is preferable that a substance breaking conformation of the modified protein 105 such as urea, surfactant is developed previously in the liquid including the modified protein 105. By this procedure, the modified protein 105 may be elongated assuredly.

For elongating the modified protein 105, shear stress may also be utilized. For example, there are a method in which the modified protein 105 is sprayed by a spray and adhered to the surface of the base plate 107, a method in which flow velocity is generated on the surface of the base plate 107, and the modified protein 105 is introduced into this and adhered to the surface of the base plate 107, and other methods. As one method for generating flow velocity, for example, the modified protein 105 may be introduced and adhered to the surface of the base plate 107 while rotating the base plate 107.

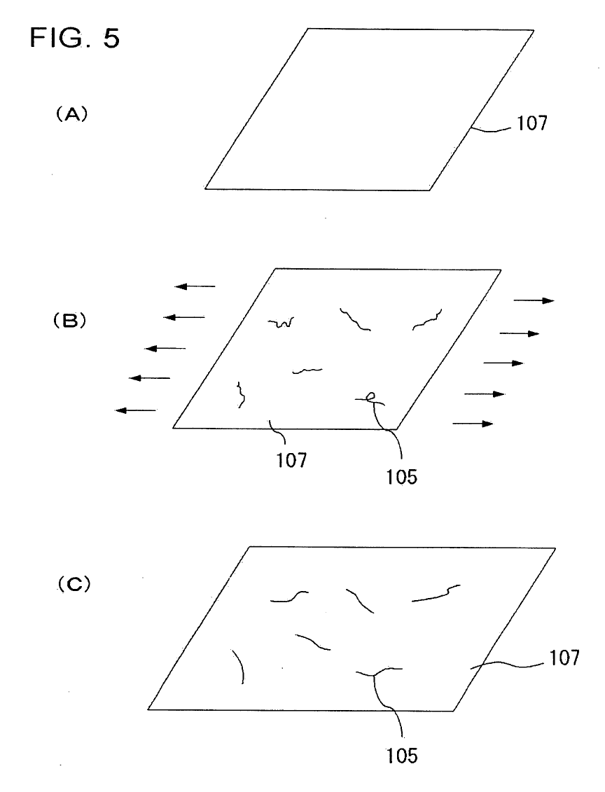

Further, the modified protein 105 may also be elongated using an elastic member as the base plate 107. FIGS. 5A to 5C are views for illustrating a method of elongating the modified protein 105.

The modified protein 105 is immobilized on the surface of the base plate 107 shown in FIG. SA (FIG. 5B). Also here, the modified protein 105 is, under elongated condition, immobilized on the surface of the base plate 107 by, for example, applying low frequency, applying high electric field, utilizing shear stress, and the like.

As shown in FIG. 5B, the base plate 107 is elongated by applying uniform force on the side of the base plate 107. By this operation, the base plate 107 is elongated and the modified protein 105 immobilized on the surface of the base plate 107 is also elongated (FIG. 5C). When the base plate 107 is thus elongated, the base plate 107 is preferably constituted of a material which uniformly elongates and does not shrink after elongation. As such material, for example, PDMS (polydimethylsiloxane) may be used. By this, the modified protein 105 may be elongated by a simple manner.

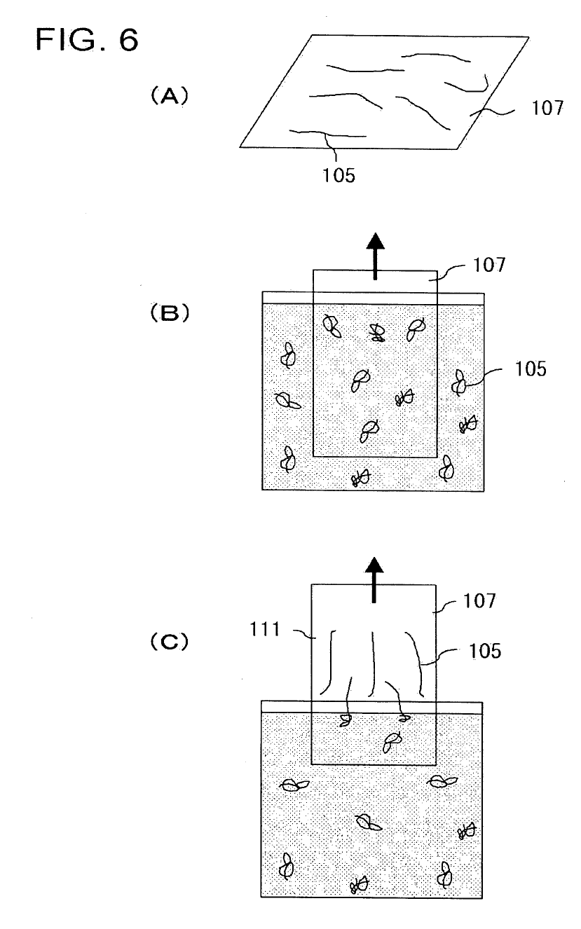

Further, for elongating the modified protein 105, meniscus force may be used. FIG. 6 is a view for illustrating a method of elongating the modified protein 105 using meniscus force. FIG. 6A is a perspective view showing a condition of elongation of the modified protein 105 on the surface of the base plate 107. The material of the base plate 107 is, for example, glass.

FIGS. 6B and FIG. 6C are cross-sectional view showing a process of elongation of the modified protein 105. First, the base plate 107 is immersed in a liquid including the modified protein 105. Then, the modified protein 105 is adsorbed on the surface of the base plate 107 (FIG. 6B). Here, the base plate 107 is lifted upward at predetermined speed. Then, the modified protein 105 adsorbed in the lifting process is elongated on the surface of the base plate 107 (FIG. 6C), to give a condition shown in FIG. 6A.

Immobilization in the step 113 may be conducted by, for example, using a base plate having a hydrophobic surface and allowing amodifiedprotein to adhere, then, drying this. On the elongated modified protein, a hydrophobic region is exposed. Therefore, immobilization may be performed easily by rendering the base plate surface hydrophobic.

When a cysteine residue of a protein is not modified with a marker 103, a gold-thiol bond may be formed via a free thiol group by allowing the surface of the base plate 107 to be made of gold.

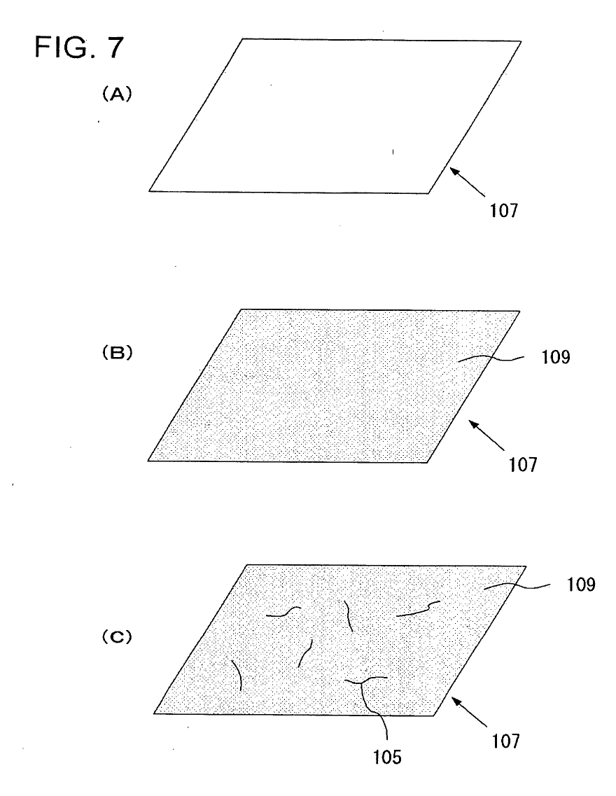

On the surface of the base plate 107, an immobilization reagent for immobilizing the modified protein 105 may be introduced previously. FIGS. 7A to 7C are views schematically showing a condition of elongation of a modified protein on a base plate carrying an introduced immobilization reagent. In this case, first, an immobilization reagent 109 is introduced on the base plate 107 shown in FIG. 7A (FIG. 7B). By developing developer containing the modified protein 105 using the above-mentioned method, the modified protein 105 may be elongated via the immobilization reagent 109 on the surface of the base plate 107, and immobilized as it is (FIG. 7C).

Washing of the step 114 is a step of washing the surface of the base plate 107 once dried, with ultra pure water and the like. By drying, the modified protein 105 is irreversibly adhered to the surface of the base plate 107, while substances such as a surfactant present in the developer are re-dissolved or re-dispersed in water. Therefore, these co-existent substances may be removed.

Again in FIG. 1, detection of the step 103 may be, in the case of use of a fluorescent substance as a marker, conducted by irradiating the base plate with a light including an excited wavelength of the fluorescent substance. By detecting by a fluorescent method, detection sensitivity may be improved. By this, the presence position of the modified protein developed on the base plate may be detected for every one molecule.

Scanning of the step 104 may be conducted, for example, by observing a surface along the shape of a modified protein by AFM (atomic force microscope) or STM (scanning tunnel microscope). Since a specific amino acid residue of a protein is modified with a marker, when scanning is effected along the primary structure of a protein, a position modified with a marker becomes bulky as compared with a position not modified, and a convex portion is formed on the upper surface or side surface of the modified protein 105. By this structure, information such as the modification position of a marker, interval between markers, and the like may be obtained by scanning an irregularity of the modified protein.

Analysis of the step 105 may be conducted referring to data base and the like based on an information relating to the modified protein obtained in the step 104. For example, since an interval between markers reflects an interval between specific amino acid residues, if a protein in which an interval between specific amino acid residues corresponds to an interval of markers is searched from a data base regarding the primary structure of a protein, its protein may be identified.

By effecting analysis in such an order, a protein can be analyzed at 1 zepto mol level.

When a protein is, for example, BSA (bovine serum albumin), a lysine residue is fluorescence-labeled with 2′,7′-Difluorofluorescein carboxylic acid succinimidyl ester in a buffer including urea, and unmodified fluorescent substances and salts are removed by dialysis. The resultant modified BSA is adhered by spraying on a glass base plate endowed with a low frequency electric field. The modified BSA adheres in the form of single chain elongated on the surface of the base plate. Thereafter, the surface of the base plate carrying the adhered modified BSA is dried. By drying the surface of the base plate, the modified BSA is irreversibly adhered and immobilized on the surface of the base plate.

The surface of the base plate is observed by a fluorescent microscope, the presence position of the modified BSA is confirmed, and irregularity of its surface is observed by AFM along a single chain of the modified BSA present on the confirmed position. Then, a part on which a fluorescent substance is adhered is observed as a convex portion. By measuring an interval between the convex portions, a value approximately equal to any of intervals between lysine residues of BSA is obtained.

Thus, according to this embodiment, it becomes possible to modify or unfold a protein as an analysis subject, specify the presence position of one molecule of the denatured protein, and perform an observation by a microscope along its skeletal chain. Therefore, analysis of high accuracy and high likelihood becomes possible regarding the primary structure of a protein, and the like.

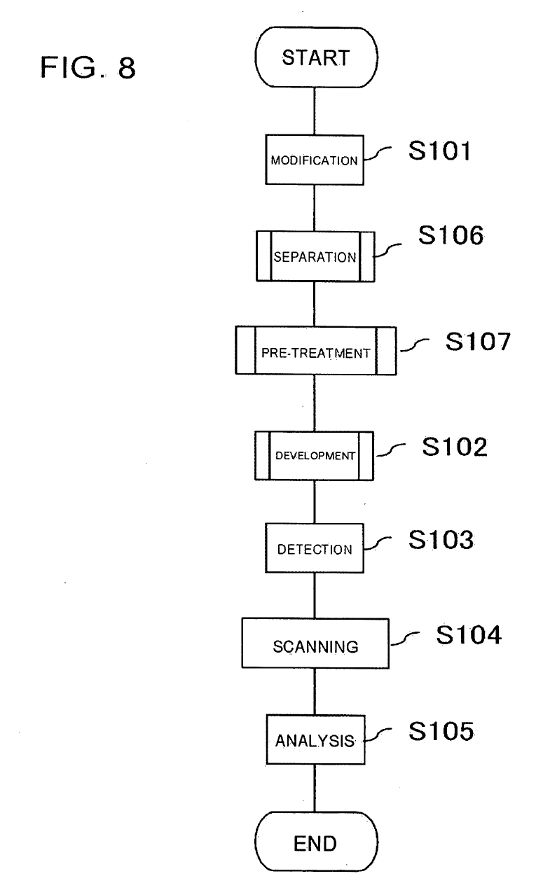

(Second Embodiment) FIG. 8 is a view showing a procedure of a method of analyzing a biomolecule according to this embodiment. The flow of FIG. 8 is different from the flow of FIG. 1 in that a plurality of components in a sample are separated (S106) following modification of the step 101 and in that a pre-treatment (S107) is conducted prior to developing (S102). As the sample, for example, a tissue extract, cell extract and the like may be used.

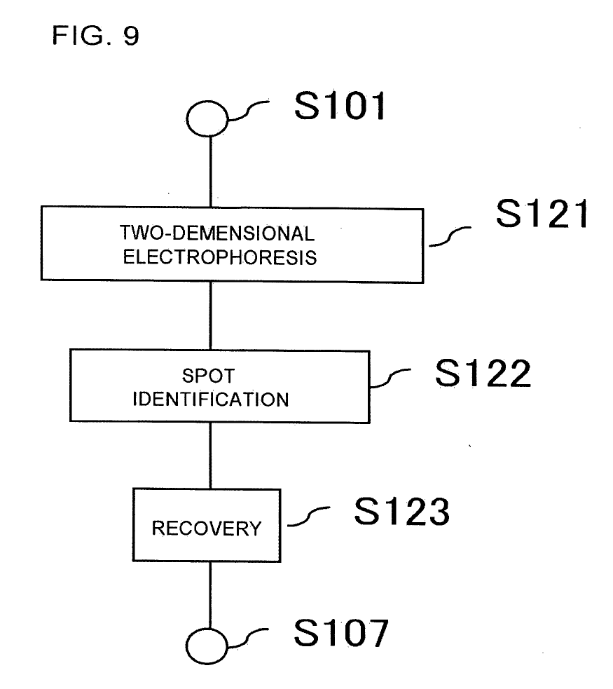

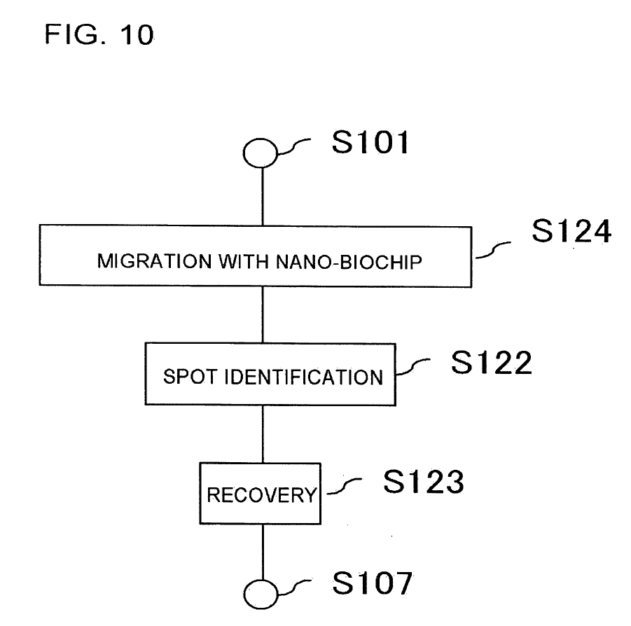

In FIG. 8, separation of the step 106 may be conducted as shown in FIG. 9 or 10. FIGS. 9 and 10 are views showing the order of the step 106 in detail.

In FIG. 9, a protein modified with a marker is identified by two-dimensional electrophoresis (S122), and the modified protein included in the intended spot is recovered (S123). The above-mentioned step may be conducted using a known method regarding two-dimensional electrophoresis of a protein. For example, when the marker is a fluorescent substance, it is possible that a protein is subjected to gel electrophoresis in two dimensions of an isoelectric point and a molecular weight, then, a light including the excited wavelength of a fluorescent substance is allowed to irradiate, and the position of a spot is identified. The modified protein may be advantageously recovered by imparting voltage along the thickness direction of the gel to elute the modified protein, or transferring it in the form of membrane, and the like. When the marker is made of a fluorescent substance, dyeing for confirming a spot becomes unnecessary, and removal of a substance used for dyeing in the subsequent step is not needed. Therefore, the procedure is simple.

FIG. 10 shows a method in which separation of a protein is conducted using a biochip instead of two-dimensional electrophoresis in FIG. 9. By using a biochip, components may be separated and recovered assuredly also when the sample is used in trace amount.

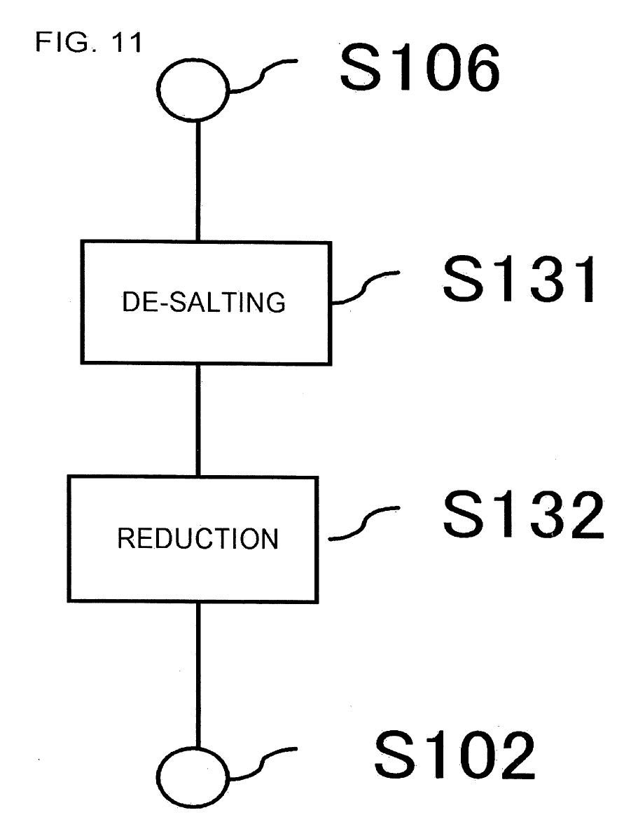

Again in FIG. 8, the pre-treatment of the step 107 may be conducted, for example, in the order of FIG. 11. FIG. 11 is a view illustrating the order of the step 107 in detail. In FIG. 11, since salts derived from the buffer are included in the separated and recovered spots, de-salting is conducted (S131).

When a disulfide bond is present in the modified protein, elongation in developing on the base plate in the subsequent step 102 is prevented in some cases. Then, a reducing agent such as DTT (dithiothreitol) is added, and a disulfide bond is reduced (S132). The reducing agent added here may be removed by washing in the above-mentioned step 114, after fixing the modified protein on the base plate.

Since the analysis method of this embodiment includes a step of separating a sample, components in a sample including a plurality of proteins may be separated and analysis may be conducted for each component. Since information such as the isoelectric point of a protein, the molecular weight thereof may be obtained by separation, the width and accuracy of analysis may be further improved by combining these information with information regarding the modified protein on the base plate.

(Third Embodiment)

In the above-mentioned embodiments, examples using a single type of marker have been illustrated, however, a plurality of markers may be used in combination. As the plurality of markers, substances which respectively specifically modify different amino acid residues of a protein as an analysis subject are used. As the analysis method using a plurality of markers, there are mentioned (i) a method in which one biomolecule is modified with a plurality of markers, and analysis is conducted using this, and (ii) a method in which analysis is conducted using a set of biomolecules modified with any of a plurality of markers. Hereinafter, these will be illustrated by turns.

(i) A method in which one biomolecule is modified with a plurality of markers, and analysis is conducted using this

In this case, substances of different size or shape are used as the plurality of markers modifying different amino acid residues. In this manner, it is possible to know which position is modified with which marker, in scanning a modified protein. Therefore, also when a sample is used in a trace amount, a lot of information regarding the arrangement of a marker may be obtained. Because of this, accuracy and likelihood of analysis may be improved.

Specifically, for example, using a fluorescent substance adsorbing or binding specifically to a lysine residue, and a fluorescent substance adsorbing or binding specifically to a cysteine residue, a lysine residue and a cysteine residue of one protein molecule may be modified simultaneously. By this procedure, the information regarding an interval between lysine residues, an interval between cysteine residues and an interval between a lysine residue and a cysteine residue, in a modified protein on a base plate, is obtained, therefore, the protein may be identified with higher accuracy.

(ii) A method in which analysis is conducted using a set of biomolecules modified with any of a plurality of markers

In this case, a sample including a protein as an analysis subject is divided into sets of the same number as the number of markers, and respective sets are modified with different markers. The resultant modified molecules are analyzed by the above-mentioned method.

By dividing a sample for every marker, each set may be modified assuredly even if amino acid residues modified by respective markers are adjacent. Further, it is not necessary that the molecular sizes of markers are different.

Specifically, for example, a sample including a protein to be analyzed is bisected previously, and one, lysine residue may be modified and another, cysteine residue may be modified. By this procedure, analysis may be performed with good accuracy even if a lysine residue and a cysteine residue are adjacent.

In this embodiment, a marker modifying the N-terminal or the C-terminal of a protein, and a marker modifying a specific amino acid residue may be used in combination. By this, the N-terminal and the C-terminal may be discriminated when a modified protein is scanned. Further, it becomes possible to obtain information regarding the positional relation of these terminals and specific amino acid residue. Because of this, likelihood and accuracy of analysis may be further improved.

Hereinafter the present invention has been illustrated based on embodiments above. These embodiments are exemplary, and those skilled in the art understand that combinations of these constituent components and treating processes may include various variants and these variants are also included in the scope of the present invention.

For example, a protein may be fractioned by a previous enzymatic treatment and the like before modifying a protein with a marker. By this operation, the length of each fragment and the presence position of a specific amino acid residue may be analyzed in combination. Because of this, a larger amount of information regarding a protein as an analysis subjected may be obtained. As a result, likelihood and accuracy of analysis may be further improved.

1. A method of analyzing a biomolecule, comprising:
elongating on a base plate a biomolecule having a specific portion selectively modified by a marker;
detecting said marker to specify a position of said elongated biomolecule on said base plate; and
analyzing an arrangement of said marker in said biomolecule.

2. The method of analyzing a biomolecule as set forth in claim 1,
wherein said analyzing the arrangement of the marker comprises measuring an interval of a plurality of said markers on said biomolecule and analyzing the arrangement of said marker.

3. The method of analyzing a biomolecule as set forth in claim 1,
wherein said elongating the biomolecule on the base plate comprises immobilizing said biomolecule on said base plate and washing the surface of said base plate on which said biomolecule has been immobilized.

4. The method of analyzing a biomolecule as set forth in claim 1,
wherein said elongating the biomolecule on said base plate comprises applying low frequency electric field on said base plate.

5. The method of analyzing a biomolecule as set forth in claim 1,
wherein said marker is a fluorescent substance.

6. The method of analyzing a biomolecule as set forth in claim 1,
wherein said specifying the position of the biomolecule on the base plate comprises irradiating a surface of said base plate with litht.

7. The method of analyzing a biomolecule as set forth in claim 1,
wherein said analyzing the arrangement of the marker comprises measuring irregularity on the surface of said biomolecule elongated on said base plate, to specify the modified position of said marker.

8. The method of analyzing a biomolecule as set forth in claim 7,
wherein said irregularity is measured by an atomic force microscope or a scanning tunnel microscope.

9. The method of analyzing a biomolecule as set forth in claim 1, further comprising separating said biomolecule, prior to said elongating the biomolecule on said base plate.

10. The method of analyzing a biomolecule as set forth in any one of claims 1 to 9 claim 1,
wherein said biomolecule is a protein or a polypeptide, and said marker modifies a specific amino acid residue of said biomolecule.

11. The method of analyzing a biomolecule as set forth in claim 10,
wherein said marker is a fluorescent substance which selectively modifies a lysine residue or a cysteine residue of said biomolecule.

12. The method of analyzing a biomolecule as set forth in claim 10, comprising denaturing said biomolecule, prior to said elongating the biomolecule on said base plate.

13. The method of analyzing a biomolecule as set forth in claim 10,
wherein said marker comprises two or more fluorescent substances of different size, and these substances modify selectively different amino acid residues in said biomolecule, respectively.

14. A method of identifying a biomolecule, comprising
analyzing a biomolecule by the method of analyzing a biomolecule as set forth in claim 1, then, further identifying said biomolecule based on an information relating to said arrangement of said marker.

15. The method of analyzing a biomolecule as set forth in claim 2,
wherein said elongating the biomolecule on the base plate comprises immobilizing said biomolecule on said base plate and washing the surface of said base plate on which said biomolecule has been immobilized.

16. The method of analyzing a biomolecule as set forth in claim 2,
wherein said elongating the biomolecule on said base plate comprises applying low frequency electric field on said base plate.

17. The method of analyzing a biomolecule as set forth in claim 3,
wherein said elongating the biomolecule on said base plate comprises applying low frequency electric field on said base plate.

18. The method of analyzing a biomolecule as set forth in claim 2,
wherein said elongating the biomolecule on said base plate comprises applying low frequency electric field on said base plate.

19. The method of analyzing a biomolecule as set forth in claim 3,
wherein said elongating the biomolecule on said base plate comprises applying low frequency electric field on said base plate.

20. The method of analyzing a biomolecule as set forth in claim 4,
wherein said elongating the biomolecule on said base plate comprises applying low frequency electric field on said base plate.
